(19) United States
(12) Patent Application Publication
(10) Pub. No.: US 2023/0041414 A1
(43) Pub. Date: Feb. 9, 2023

(54) STABILIZATION OF OXIDASES BY DRYING UNDER REDUCED PARTIAL OXYGEN PRESSURE

(71) Applicant: DANISCO US INC, Palo Alto, CA (US)

(72) Inventors: ANGELA CIFELLI, BOULDER CREEK, CA (US); PEYMAN MOSLEMY, SUNNYVALE, CA (US)

(21) Appl. No.: 17/786,720

(22) PCT Filed: Dec. 18, 2020
(86) PCT No.: PCT/US2020/066071

§ 371 Date: Jun. 17, 2022

Related U.S. Application Data
(60) Provisional application No. 62/949,557, filed on Dec. 18, 2019.

(51) Int. Cl. C12N 9/04

(52) U.S. Cl. CPC C12N 9/0006; C12Y 101/03004; C12Y 101/03005

(57) ABSTRACT

Described are compositions and methods relating to the stabilization of oxidase enzymes by heat (or thermal) drying under reduced partial pressure of diatomic oxygen. The compositions and methods allow for dehydration of oxidase-containing compositions with no loss of enzyme activity.

### CROSS REFERENCE

This application is a 371 of International Application No. PCT/US2020/066071 filed Dec. 18, 2020, which claims the benefit of U.S. Patent Application No. 62/949,557, filed Dec. 18, 2019, all of which are incorporated by reference in their entirety.

### FIELD OF THE INVENTION

The present compositions and methods relate to the stabilization of oxidase enzymes by heat (or thermal) drying under reduced partial pressure of diatomic oxygen. The compositions and methods allow for drying of oxidase-containing compositions with no loss of enzyme activity.

### BACKGROUND

Oxidases (EC 1.1.3) are enzymes that catalyze oxidation-reduction reactions, typically involving diatomic oxygen (02) as an electron acceptor. Examples of oxidases are glucose oxidase, hexose oxidase, monoamine oxidase, xanthine oxidase, L-gulconolactone oxidase, lysyl oxidase, NADPH oxidase, polyphenol oxidase, cytochrome P450 oxidase and laccase. Numerous additional enzymes are listed, herein.

Glucose oxidase (GOx; EC 1.1.3.4) is an oxidase that can be over-expressed in heterologous hosts for large-scale production and has a particularly broad range of commercial uses. GOx, sometimes referred to as GOD, is widely used to control microbial contamination, e.g., in wine making, and in biochemical assays and biosensors to measure free glucose, e.g., in blood and urine. GOx is also used to produce stronger dough in baking, to remove oxygen in food packages, and to prevent the browning of certain foods, such as egg whites.

Hexose oxidase (HOx: EC 1.1.3.5) catalyzes the oxidation of mono- and disaccharides to their corresponding lactones, with concomitant reduction of molecular oxygen to hydrogen peroxide. This enzyme is produced commercially by over-expression in certain methylotrophic yeasts. Hexose oxidase is able to oxidize a variety of substrates including D-glucose, D-galactose, maltose, cellobiose, and lactose. The wide substrate specificity distinguishes this enzyme from GOx which is highly specific for D-glucose. HOx is also used to produce stronger dough in baking.

GOx and HOx are sensitive enzymes and their commercial production is made expensive and inefficient by considerable activity loss during production and storage. Dehydration processes such as a spray drying that involve desiccation via heating are particularly destructive to the enzyme proteins. In view of the myriad uses of GOx and HOx, the need exists for ways to increase stabilization in a cost-effective manner.

### SUMMARY

The present compositions and methods relate to the stabilization of oxidases by heat (or thermal) drying under reduced partial pressure of oxygen conditions compared to atmospheric conditions. The compositions and methods allow for dehydration of oxidase-containing compositions with no loss of enzyme activity. Aspects and embodiments of the compositions and methods are described in the following, independently-numbered paragraphs.

1. In one aspect, a method for increasing the recovery of oxidase enzyme activity in a dried oxidase enzyme composition is provided, comprising; thermal drying an oxidase enzyme in the presence of less than normal atmospheric partial oxygen pressure conditions, wherein, upon reconstitution of the dried oxidase enzyme in an aqueous solution or suspension, the oxidase enzyme dried under the less than a normal atmospheric partial oxygen pressure conditions exhibits increased activity compared to the same oxidase enzyme dried under normal atmospheric partial oxygen pressure conditions, wherein normal atmospheric partial oxygen pressure conditions are measured at normal temperature and pressure.

2. In some embodiments of the method of paragraph 1, the partial pressure of oxygen under which the oxidase enzyme is dried does not exceed 120 mm-Hg.

3. In some embodiments of the method of paragraph 2, the partial pressure of oxygen under which the oxidase enzyme is dried does not exceed 80 mm-Hg.

4. In some embodiments of the method of paragraph 3, the partial pressure of oxygen under which the oxidase enzyme is spray-dried does not exceed 40 mm-Hg.

5. In some embodiments of the method of any of paragraphs 1-4, thermal drying of the oxidase enzyme is performed under vacuum.

6. In some embodiments of the method of any of paragraphs 1-4, thermal drying of the oxidase enzyme is performed under an inert gas.

7. In some embodiments of the method of paragraph 6, thermal drying of the oxidase enzyme is performed under nitrogen.

8. In some embodiments of the method of any of paragraphs 1-7, the oxidase enzyme is glucose oxidase or hexose oxidase.

9. In another aspect, an oxidase enzyme produced by the method of any of paragraphs 1-8 is provided.

These and other aspects and embodiments of the compositions and methods will be apparent from the present description.

### DETAILED DESCRIPTION OF THE INVENTION

### I. Introduction

The inventors have discovered that heat (or thermal) drying oxidase enzymes under condition of reduced partial pressure of molecular oxygen, improves the activity of the enzyme upon reconstitution in an aqueous solution or suspension. Increasing the stability, or recovered activity, of an oxidase by controlling the partial pressure of oxygen under conditions for preparing the enzyme for storage, has heretofore not been described.

### II. Definitions and Abbreviations

Prior to describing the present compositions and methods in detail, the following terms are defined for clarity. Terms not defined should be accorded their ordinary meanings as used in the relevant art.

As used herein, interchangeably, “heat drying” or “thermal drying” is a process for the dehydration of an aqueous enzyme composition based on water evaporation by heating into a solid composition of the said enzyme.

As used herein, the term “granule” refers to a small particle of a substance. The particle comprises a core, optionally with one or more coating layers.

As used herein, the term “recovered activity” or “activity recovery” refers to the ratio of (i) the activity of an enzyme after a treatment involving one or more of the following stressors: heating, increased pressure, increased pH, decreased pH, storage, drying, exposure to surfactant(s), exposure to solvent(s), and mechanical stress) to (ii) the activity of the enzyme before the treatment. The recovered activity may be expressed as a percentage. The percent recovered activity is calculated as follows:

$$\% recovered activity = \frac{( activity after treatment )}{( activity before treatment )} \times 100 \%$$

As used herein, “normal temperature and pressure” is 20° C. and 1 atm.

As used herein, the term “about” refers to ±15% to the referenced value.

For ease of reference, elements of the present compositions and methods may be arranged under one or more headings. It is to be noted that the compositions and methods under each of the headings also apply to the compositions and methods under the other headings.

As used herein, the singular articles “a,” “an” and “the” encompass the plural referents unless the context clearly dictates otherwise. All references cited herein are hereby incorporated by reference in their entirety. The following abbreviations/acronyms have the following meanings unless otherwise specified:
° C. degrees Centigrade
atm atmosphere
g or gm gram
g/L grams per liter
g/mol grams per mole
mol/mol mole to mole ratio
μmol micromole
hr or h hour
kg kilogram
mg milligram
mL or ml milliliter
min minute
M molar
mM millimolar
micrometer (micron)
UFC ultrafiltered concentrate
dissolved solids active oxidase protein plus other non-oxidase fermentation solids
wt weight
μL and μl microliter
% wt/wt weight percent

### III. Oxidases

The discovery that oxidases can be stabilized by thermal drying under conditions of reduced partial pressure of diatomic oxygen for exemplified oxidase enzymes, is expected to apply to a broad range of oxidases. Moreover, testing the ability of such a method for stabilizing a particular oxidase is routine and does not require undue experimentation.

Particular oxidases are those that use molecular oxygen (O₂) as an acceptor, and are classified as EC 1.1.3. Exemplary oxidases include those listed in Table 1.

TABLE 1
EC 1.1.3 enzymes

| Enzyme classification | Common name |
|---|---|
| EC 1.1.3.3 | malate oxidase |
| EC 1.1.3.4 | glucose oxidase |
| EC 1.1.3.5 | hexose oxidase |
| EC 1.1.3.6 | cholesterol oxidase |
| EC 1.1.3.7 | aryl-alcohol oxidase |
| EC 1.1.3.8 | L-gulonolactone oxidase |
| EC 1.1.3.9 | galactose oxidase |
| EC 1.1.3.10 | pyranose oxidase |
| EC 1.1.3.11 | L-sorbose oxidase |
| EC 1.1.3.12 | pyridoxine 4-oxidase |
| EC 1.1.3.13 | alcohol oxidase |
| EC 1.1.3.14 | catechol oxidase (dimerizing) |
| EC 1.1.3.15 | (S)-2-hydroxy-acid oxidase |
| EC 1.1.3.16 | ecdysone oxidase |
| EC 1.1.3.17 | choline oxidase |
| EC 1.1.3.18 | secondary-alcohol oxidase |
| EC 1.1.3.19 | 4-hydroxymandelate oxidase |
| EC 1.1.3.20 | long-chain-alcohol oxidase |
| EC 1.1.3.21 | glycerol-3-phosphate oxidase |
| EC 1.1.3.23 | thiamine oxidase |
| EC 1.1.3.24 | L-galactonolactone oxidase |
| EC 1.1.3.27 | hydroxyphytanate oxidase |
| EC 1.1.3.28 | nucleoside oxidase |
| EC 1.1.3.29 | N-acylhexosamine oxidase |
| EC 1.1.3.30 | polyvinyl-alcohol oxidase |
| EC 1.1.3.37 | D-arabinono-1,4-lactone oxidase |
| EC 1.1.3.38 | vanillyl-alcohol oxidase |
| EC 1.1.3.39 | nucleoside oxidase (H2O2-forming) |
| EC 1.1.3.40 | D-mannitol oxidase |
| EC 1.1.3.41 | xylitol oxidase |
| EC 1.1.3.42 | prosolanapyrone-II oxidase |
| EC 1.1.3.43 | paromamine 6′-oxidase |
| EC 1.1.3.44 | 6-hydroxyneomycin C oxidase |

The oxidase enzyme is stabilized in a dry mixture prepared by heating. The stabilized oxidase may be incorporated into a dry granule or other solid composition, wherein reconstitution in an aqueous solution or suspension results in increased oxidase activity compared to that of the same oxidase dried under the same conditions at normal oxygen partial pressure at normal temperature and pressure.

### IV. Partial Pressure of Oxygen

It is likely that different oxidases will require different reduced partial pressures of oxygen for stabilization upon drying, which amounts are readily determined. Such amounts are best expressed in terms of partial pressure, which is the theoretical pressure of the oxygen component of a gas if it alone occupied the entire volume of the original gas mixture at the same temperature.

Air is typically 21% oxygen and atmospheric pressure is 760 mm Hg (at sea level). Therefore, the partial pressure of oxygen is 0.21×760 mm Hg=160 mm Hg. Reducing the partial pressure of oxygen in the air can generally be achieved by one of several methods, or a combination of methods. The first is by reducing the pressure of the entire gas (typically air) to which the oxidase is exposed during thermal drying. This can be achieved by drying under vacuum. A second is to selectively adsorb oxygen from the gas to which the oxidase is exposed during drying. This can be achieved by cryogenic air separation or pressure swing adsorption. A third option is to dry the oxidase under an inert gas, such as nitrogen, or periodic table group 18 elements.

Regardless of the method used, preferably, the partial pressure of oxygen to which the oxidase is exposed during drying is no more than 120, no more than 100, no more than 80, no more than 60, or even no more than 40, or fewer mm-Hg.

### EXAMPLES

### Example 1. Thermal Inactivation of Glucose Oxidase (GOx) Upon Thermal Drying Under Atmospheric Pressure

Two samples of a neat (unformulated) GOx UFC (25 mg active protein per g UFC) were obtained from DuPont/Danisco fermentation plants. The samples were incubated in an oven at 50° C. for four, eight and twenty-four hours under normal atmospheric pressure to dehydrate into a dry composition. The experimental method involved addition of 80 μL of each sample to interior wells of a 96-well plate (to avoid edge effects), sealing the plate with breathable seal, and incubating the plate at 50° C. The weight of the plate was recorded before and after drying to ensure all water was removed.

To measure activity, the dried composition of each well was re-suspended in 80 μL of purified water. After resuspension, the activity was measured using a standard glucose oxidase assay. Glucose oxidase catalyzes the conversion of glucose and oxygen to hydrogen peroxide and gluconic acid in the assay. A reaction of hydrogen peroxide with 2,2′-azino-bis(3-ethylbenzthiazolin-6-sulfonsyre), ABTS, changes the appearance of reaction media from colorless to green. This reaction is catalyzed by the enzyme peroxidase. The green color is measured on a spectrophotometer at 405 nm. The method is calibrated using a linear regression of standard dilutions prepared from a standard material with a known concentration. The oxidase enzymatic activity decreased with time as shown in Table 2.

TABLE 2
Oxidase activity yields of neat GOx UFC samples
incubated at 50° C. under atmospheric pressure
for 4, 8, and 24 hours; n = 8, avg. ± stdev

| Unformulated | % Recovered Activity | | |
|---|---|---|---|
| GOx UFC | 4 hr | 8 hr | 24 hr |
| Sample #1 | 81% ± 2.8% | 52% ± 3.4% | 31% ± 3.6% |
| Sample #2 | 71% ± 4.8% | 34% ± 7.4% | 13% ± 11% |

### Example 2. Increase in Recovered Activity of GOx Upon Thermal Drying Under Vacuum, Reduced Partial Pressure of Oxygen

The neat GOx UFC samples described in Example 1 were incubated in an oven at 50° C. for four, eight and twenty-four hours under vacuum at 760 mm-Hg to dehydrate into a dry composition. The experimental setup and method for analysis of activity were similar to those described in Example 1. The recovered enzyme activity of dried samples was, surprisingly, higher than that of the sample before drying as shown in Table 3.

TABLE 3
Oxidase activity yields of neat GOx UFC samples
incubated at 50° C. under vacuum at
760 mm-Hg for 4, 8, and 24 hours; n = 8, avg. ± stdev

| Unformulated | % Recovered Activity | | |
|---|---|---|---|
| GOx UFC | 4 hr | 8 hr | 24 hr |
| Sample #1 | 122% ± 6.80% | 125% ± 18.4% | 184% ± 16.6% |
| Sample #2 | 132% ± 8.46% | 173% ± 12.4% | 180% ± 14.0% |

### Example 3: Variation of Recovered Activity of GOx with Partial Pressure of 02 in Thermal Drying

As shown in the previous example, GOx compositions exhibited higher activity when dried at 50° C. under vacuum at 760 mm-Hg. The present experiment was conducted under different vacuum pressures of 0, 380 and 760 mm-Hg, respectively corresponding to oxygen partial pressures of 160, 80, and 0 mm-Hg, to further study the effect of atmospheric oxygen on enzyme activity yield. The neat GOx UFC samples described in Example 1 were incubated at 50° C. for four hours under vacuum pressures of 0, 380 and 760 mm-Hg to dehydrate into a dry composition. The experimental setup and method for analysis of activity were similar to those described in Example 1. The oxidase enzymatic activity increased with increased vacuum as shown in Table 4.

TABLE 4
Oxidase activity yields of neat GOx UFC samples incubated
under vacuum at 50° C. for 4 hours; n = 8, avg. ± stdev

| | % Recovered Activity | | |
|---|---|---|---|
| Unformulated | 0 mm-Hg | 380 mm-Hg | 760 mm-Hg |
| GOx UFC | vacuum | vacuum | vacuum |
| Sample #1 | 76% ± 9.2% | 99% ± 17% | 119% ± 8.26% |
| Sample #2 | 69% ± 8.3% | 74% ± 6.2% | 106% ± 8.20% |

### Example 4. Increase in Recovered Activity of GOx and Hexose Oxidase (HOx) Upon Thermal Drying Under Nitrogen Compared to Atmospheric Oxygen

One sample of a neat GOx UFC (25 mg active protein per g UFC) and one sample of HOx UFC (7.2 mg active protein per g UFC) were obtained from DuPont/Danisco fermentation plants. The samples were incubated in an oven at 50° C. for four hours under atmospheric pressure to dehydrate into a dry composition. The experimental setup under atmospheric oxygen (air) and method for analysis of activity were similar to those described in Example 1. The same set up was employed for drying with pure nitrogen gas (N₂). The nitrogen gas, sourced from a compressed gas tank, was purged through the oven at near atmospheric pressure. The samples dried under nitrogen exhibited higher retained activity compared to those dried under atmospheric oxygen pressure (i.e., as in air) as shown in Table 5.

TABLE 5
Oxidase activity yields of neat GOx and hexose oxidase UFC
samples incubated at 50° C. for 4 hours; n = 4, avg. ± stdev

| | % Recovered Activity | |
|---|---|---|
| Unformulated UFCs | GOx | HOx |
| Atmospheric oxygen (air) | 10% ± 1.2% | 27% ± 0.7% |
| Nitrogen, ultrapure | 57% ± 7.1% | 72% ± 4.2% |

What is claimed is:

1. A method for increasing the recovery of oxidase enzyme activity in a dried oxidase enzyme composition, comprising; thermal drying an oxidase enzyme in the presence of less than normal atmospheric partial oxygen pressure conditions, wherein, upon reconstitution of the dried oxidase enzyme in an aqueous solution or suspension, the oxidase enzyme dried under the less than a normal atmospheric partial oxygen pressure conditions exhibits increased activity compared to the same oxidase enzyme dried under normal atmospheric partial oxygen pressure conditions, wherein normal atmospheric partial oxygen pressure conditions are measured at normal temperature and pressure.

2. The method of claim 1, wherein the partial pressure of oxygen under which the oxidase enzyme is dried does not exceed 120 mm-Hg.

3. The method of claim 2, wherein the partial pressure of oxygen under which the oxidase enzyme is dried does not exceed 80 mm-Hg.

4. The method of claim 3, wherein the partial pressure of oxygen under which the oxidase enzyme is spray-dried does not exceed 40 mm-Hg.

5. The method of any of claims 1-4, wherein thermal drying of the oxidase enzyme is performed under vacuum.

6. The method of any of claims 1-4, wherein thermal drying of the oxidase enzyme is performed under an inert gas.

7. The method of claim 6, wherein thermal drying of the oxidase enzyme is performed under nitrogen.

8. The method of any of claims 1-7, wherein the oxidase enzyme is glucose oxidase or hexose oxidase.

9. An oxidase enzyme produced by the method of any of claims 1-8.
